(19) United States
(12) Patent Application Publication
(10) Pub. No.: US 2011/0117554 A1
(43) Pub. Date: May 19, 2011

(54) Formamide-containing mixtures for detecting nucleic acids

(76) Inventors: Steven Albert Benner, Gainesville, FL (US); Daniel Hutter, Gainesville, FL (US); Nicole A. Leal, Gainesville, FL (US)

(21) Appl. No.: 12/927,430

(22) Filed: Nov. 15, 2010

Related U.S. Application Data
(60) Provisional application No. 61/281,319, filed on Nov. 16, 2009.

(51) Int. Cl. C12Q 1/68

(52) U.S. Cl. 435/6

(57) ABSTRACT

Specific sequences of DNA are often detected by a process that comprises a step where the sequence to be detected (the “analyte”) binds to give a duplex with a DNA molecule or analog that is complementary in the Watson-Crick sense to some portion of the analyte in an aqueous “assay environment” that may contain buffer, salt, and/or detergent. Such purely aqueous systems cannot be exposed indefinitely to the environment, however, as the water in the system will evaporate. Further, such systems often support the growth of bacteria and other organisms, destroying their effectiveness. This invention provides for compositions of matter and processes that use them that comprise assay mixtures containing more than 40% formamide. This mixture remains a liquid at equilibrium with water in environments normally inhabited by humans. This invention also provides for mixtures containing formamides that include detergents. Formamide is usually regarded as a denaturant for duplex formation, destabilizing the binding that is key to detection. This invention therefore also provides for materials that form duplexes in formamide-water mixtures.

### CROSS REFERENCE TO RELATED APPLICATIONS

This Application claims priority to U.S. Provisional Patent Application No. 61/281,319, filed Nov. 16, 2009

### STATEMENT REGARDING FEDERALLY SPONSORED RESEARCH OR DEVELOPMENT

None

### THE NAMES OF THE PARTIES TO A JOINT RESEARCH AGREEMENT

Not applicable

### INCORPORATION-BY-REFERENCE OF MATERIAL SUBMITTED ON A COMPACT DISC

None

### BACKGROUND OF THE INVENTION

(1) Field of the Invention

This application relates to compositions of matter that support assays for detecting nucleic acids in mixtures that may be continuously exposed to an environment whose humidity is less than 100%, whereby the assay occurring in the mixture having the composition of the instant invention involves the binding of a nucleic acid or analog to a complementary DNA or RNA molecule that is being detected by the assay.

(2) Description of Related Art

Nucleic acids are used in many schemes as probes to detect nucleic acid (DNA or RNA) analytes provided by pathogens or other living species. They are also used to detect fully synthetic nucleic acids and their analogs. In most cases, an early step in that detection process comprises the contacting of a probe that is itself a nucleic acid or analog with the analyte to form a duplex between the two, where the specificity of duplex formation is determined by Watson-Crick binding rules (A pairs with T or U, G pairs with C, and, for expanded genetic alphabets, the analogous base pairs are formed).

Then, in ways determined by the assay architecture [Nam et al., 2004], which can be done in many ways, including using radioactive labels or non-radioactive labels, the duplex is detected. For example without limitation, the duplex might serve as a substrate for a DNA polymerase, supporting a polymerase chain reaction (PCR). For example without limitation, duplex formation might be part of a capture step that moves a fluorescent moiety into a detection zone. Nearly always, however, this analysis must be done in a liquid medium, even in architectures where the duplex is ultimately immobilized on a solid support.

In general, but especially for detecting DNA in environmental samples (for example without limitation, DNA from bacteria present in the air in a post office where the humidity is less than 100%), high cost makes the detection of DNA analytes impractical. This is because each downstream step to detect the duplex adds cost. More practical is an assay system that can provide continuous surveillance of a work space for specific nucleic acids, for example, those arising from a pathogen (such as influenza). Most desirable would be an assay mixture that creates a detectable signal when it encounters the analyte nucleic acid without any downstream processing steps at all.

Here, the need to operate in a liquid creates a further constraint. Hybridization of complementary nucleic acid strands is usually done in water, often in water containing salts and buffers. Because water evaporates when the humidity is less than 100%, aqueous assay mixtures cannot be exposed indefinitely to an environment such as open atmosphere. Accordingly, assays that use aqueous assay environments require a first step, such as opening a closed container holding the assay mixture, allowing any analyte present to contact that mixture, and then closing the container. This in itself creates cost, and prevents continuous surveillance.

It would therefore be desirable to have a nucleic acid analyte detection assay mixture in a liquid that does not completely evaporate when exposed to an environment where the humidity is less than 100%, in which nucleic acid hybridization and duplex formation can occur. Even more desirable would be to arrange to have that assay mixture be homogenous and self-sterilizing. A homogenous, environmentally stable, self-sterilizing liquid that lyses virions and/or bacterial cells and supports nucleic acid hybridization would allow the hybridization of probes specific for pathogens, in particular with single nucleotide discrimination specificity.

Many non-water solvents remain liquid even upon exposure to an environment typical of those where humans live and work (from just below 0° C. to 4.0° C., relative humidity between 30% and 100%). For example, dipolar aprotic solvents such as formamide, sulfolane, and dimethylsulfoxide have high boiling points and low vapor pressure under conditions where humans normally live and work.

However, it is widely believed in the art, and this belief is well supported by experiment, that DNA duplex formation is difficult or impossible in these non-water solvents. For example, formamide is routinely used in molecular biology to destroy duplexes between DNA molecules, in a process often called “denaturation” [Blake & Delcourt, 1996] [Hutton, 1977] [Jungmann et al., 2008] [Steger, 1994]. For example, formamide is routinely added to gels that are used to electrophoretically separate nucleic acids, as it drives DNA duplexes to form single strands [Spohr et al., 1976]. This is antithetical to the duplex formation that is the essence of the nucleic acid assay architectures.

Over 30 years ago, RNA:RNA duplexes were reported to be more stable in formamide than DNA:DNA duplexes [Casey, 1977] [Chien & Davidson, 1978]. The HDV ribozyme works in 95% formamide, an activity that the literature teaches “is unique”, and is believed to relate to a particularly stable folded structure for the ribozyme [Duham, 1996]. However, despite this prior art, hybridization in formamide and other dipolar aprotic solvents is deliberately avoided in the art, is unexpected in the cases where it is occasionally observed, and is not used in any assays, to the best of the Applicant's knowledge and belief.

### BRIEF SUMMARY OF THE INVENTION

This invention provides liquid assay mixtures that are compositions of matter containing between 40% to 80% by volume formamide, with the remainder of the liquid volume being 20% to 60% water. These can be exposed to ambient conditions where humans live and work, where they remain liquid indefinitely by being in equilibrium with air of typical humidity. This is possible by having formamide-water mixtures that are in equilibrium with the atmospheric moisture, and where the fraction of formamide remains in this range indefinitely. The compositions may also contain a detergent such as sodium dodecyl sulfate (SDS), buffers and salts, and one or more nucleic acids or nucleic acid analogs, where one or more of these nucleic acids or nucleic acid analogs is substantially (50% or more) in the form of a duplex over part of its length (at least 10 nucleotides) with its complement.

### BRIEF DESCRIPTION OF THE SEVERAL VIEWS OF THE DRAWING(S)

FIG. 1. Fraction of formamide present at indicated temperature as a function of humidity, established by exposing a closed atmosphere to a saturated solution of the indicated salt.

FIG. 2. Twelve non-standard nucleobases in a nucleic acid alphabet that form specific pairs with the constraints of the Watson-Crick geometry but without cross reaction with standard nucleobases. Pyrimidine analogs are designated “py”, purine by “pu”. Upper case letters following a designation indicate the hydrogen bonding pattern of acceptor (A) and donor (D) groups [Benner, 2004]. As known in the art, M may be selected from the group consisting of N and C-tag, where said tag is selected from the group consisting of fluor, biotin, and alkyl.

### DETAILED DESCRIPTION OF THE INVENTION

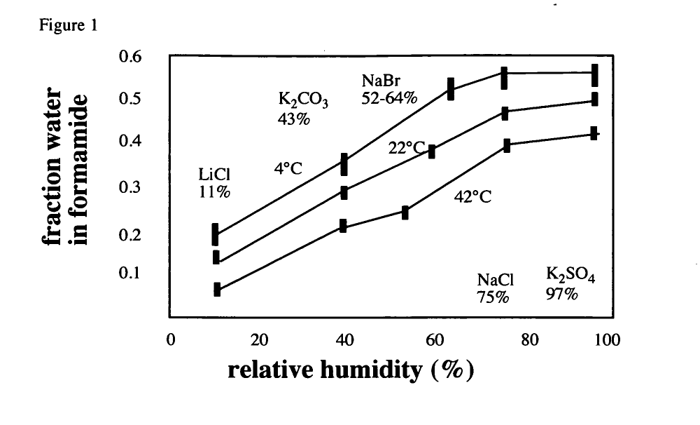

The instant invention is based on the discovery that certain nucleic acid analogs can bind to their DNA and RNA counterparts using Watson-Crick pairing in formamide-water mixtures where the fraction of formamide is high, but not 100%, but rather in the range that is in stable equilibrium with air at temperatures, pressures, and humidities where humans live and work, and therefore with air where environmental sampling is most useful (FIG. 1, Example 2).

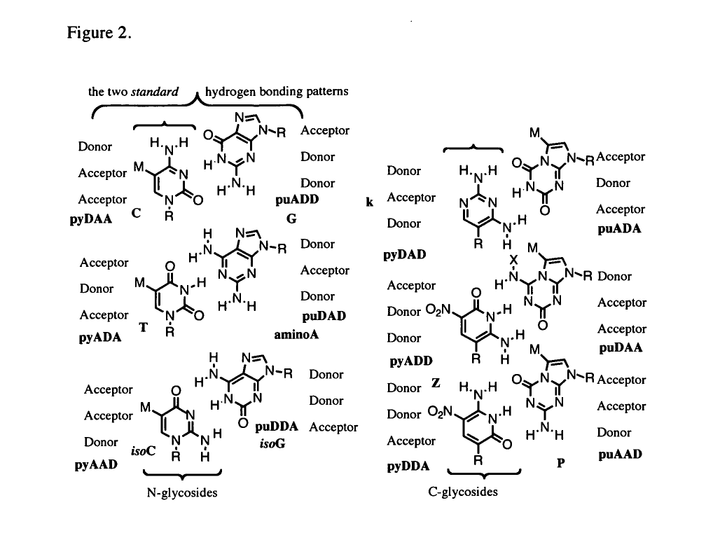

The inventive step is first the recognition that this combination allows useful duplex formation even though formamide, to those knowledgeable in the art, denatures duplexes. Also inventive was the discovery, by experiment, that adding detergent (0.2-5%) causes cells that fall into formamide-water mixtures in ratios that are stable under those conditions, lyse to expose their DNA (Example 1). Also inventive is the teaching that duplex stability can be achieved with oligonucleotides where the background sugar is 2′-O-methyl ribose. Also inventive is the teaching that duplex stability can be achieved with oligonucleotides are joined by one or more of the non-standard nucleobases shown in FIG. 2.

Formamide-water mixtures can exist as stable liquids over a wide range of temperatures, from below 0° C. to over 200° C. The low temperature range is extended by addition of water, which serve as an antifreeze for formamide. Therefore formamide-water solutions can be exposed to the environment indefinitely without drying out, even in environments where the humidity (with expect to water) is less than 100%). In these environments, the composition of mixtures oscillates as humidity changes within the range shown in FIG. 1.

As a practical matter, standard DNA, having a 2′-deoxyribose backbone and adenine as one of the four nucleobases, cannot be used in such mixtures as a probe for DNA analytes. As is shown in Table 1, collected experimentally in this work, the model 20 mer does not have a melting temperature above 20° C. in mixtures containing 70% formamide. As is well understood in the art, probes can be made longer. However, as probes become longer, they become less able to discriminate between perfectly matched complements and complements containing one or a few mismatches.

Thus, the presently preferred analogs that actually can function in formamide-water mixtures that are stable when exposed to human-habitable environments are those having an —OH or an —OR group at the 2′-position, and/or that have adenine replaced by 2,6-diaminopurine. While RNA probes have a heteroatom at the 2′-position, the 2′-OH group also makes them chemically unstable and expensive to synthesize. Therefore, the presently preferred backbone sugar is a 2′-O methyl, alkyl, or allyl ribose. The discovery that such nucleic acid analogs permit DNA- and RNA-targeted molecular recognition in formamide-water mixtures stable in human-habitable environments provides a new technical capability that has not previously been available to support assays that detect pathogen nucleic acids.

Moreover, detergents can be introduced into formamide-water mixtures without disrupting these duplexes. This makes the mixtures self-sterilizing, by which it is meant that virions and/or cells that encounter these mixtures are broken open, thereby exposing intracellular nucleic acids to detection by probes in the mixture. This also permits sample preparation that does not require the sequential addition of reagents or any thermal cycling. In principle, a pathogenic cell that falls into a formamide-water mixture containing detergents simply lyses, presenting its nucleic acid targets to the bulk solution. Anionic detergents also do not disrupt the formation of duplexes between complementary strands in formamide-water mixtures.

Therefore, this discovery allows assays to proceed in formamide-water mixtures while still promoting hybridization of nucleic acid analogs as part of an unnatural genetic system, thereby solving two of the three tasks for pathogen analyte detection (lysis and target binding). The assay simply requires exposing a prepared assay mixture to an environmental sample.

Many architectures may complete an assay in the mixtures of the instant invention. For example, the architecture might generate a signal based on hybridization between probes introduced in the formamide-water mixtures and target nucleic acid analytes from the disrupted pathogen cell. It is well-known that such signals can be generated by sandwich assays [Benner, 2004], including those that immobilize dendrimers and those that immobilize nanocrystals that change the color of their fluorescent emission when assembled in proximity [Nam et al., 2004].

This requires therefore that the innovative chemistries available to artificial genetic systems address issues related to background noise. Such chemistry must recognize the potential presence of a wide range of other non-target nucleic acids in the sample. Here, we introduce a third innovative feature arising from the existence of expanded genetic alphabets, the ability to do nucleic acid capture without interference from natural DNA or RNA. This is based on the artificially expanded genetic information system (AEGIS), a DNA analog where non-standard pairs replace A, T. G, and C in some (but not necessarily all) of the constituent nucleotides. Several of these non-standard nucleobases are shown in FIG. 2. Several AEGIS base pairs, including those between isoC and isoG and between Z and P, are more stable than standard G:C base pairs, and also support duplex formation in formamide-water mixtures that are stable when exposed to human-habitable environments.

For binding in formamide-containing assay mixtures, the presently preferred probe is 2′-β-methyl ribonucleosides. This is stable to nuclease degradation, and stable indefinitely in formamide-water mixtures. The impact of a molecular recognition system that can work in an environmentally stable, self-sterilizing homogenous fluid mixture to generate signals specific for the nucleic acid sequences of specific pathogens is obvious. The extent of that impact will depend only on the ultimate sensitivity of the system. Obviously, the greatest impact would be if a single pathogenic cell could be detected by a color change in formamide-water mixtures using a sandwich assay and an amplification architecture based on AEGIS. In some cases, especially if catalysis is desired from the nucleotide probe, a catalytic ribonucleoside is preferred.

### Example 1

### Experiments to Determine Whether Formamide Alone or with Detergent Lyses E. Coli

A strain of E. coli (TG1 or NAL3) was suspended in a mixture of 50% formamide-50% water in the presence and absence of sodium dodecyl sulfate (SDS). Following lysis, the ability to the suspension to generate colonies (colony forming units) was measured by plating on a standard agarose plate. Separately, the DNA was recovered via ethanol precipitation.

Specifically, the TG1 strain was from Zymo (NAL3): F′traD36 lacIqΔ(lacZ) M15 proA+B+/supE Δ(hsdM-mcrB)5 (rk-mk-McrB-) thi Δ(lac-proAB) LB medium (5 mL) was inoculated with an overnight culture of NAL3 (50 μL) and incubated in a New Brunswick incubator at 37° C., 250 rpm to an OD₆₀₀ of 0.92. An aliquot (800 μL) was removed and centrifuged for 3 min at 16,100×1000 rcf. The supernatant was removed and the pellet was washed once with 0.85% sterile saline solution (800 μL). Aliquots (100 μL) were placed into 7 microcentrifuge tubes, and centrifuged for 3 min 16.1×1000 rcf, The supernatant was removed and the pellet was added to and aliquot (100 μL) of the appropriate lysis buffer. The samples were rocked gently to mix (˜2 min) and incubated at room temperature for 5 min.

After lysis, the samples were centrifuged at 16.1×1000 rcf for 3 min, the supernatant was removed, and the DNA was recovered by ethanol precipitation. To the pellet was added 0.85% sterile saline (100 μL). The material was plated on LB agar. Results are collected in Table 2.

TABLE 2
Colony forming units from TG-1 (NAL3)

| Conditions | Formamide | Water | Dilution | colonies | cfu/mL |
|---|---|---|---|---|---|
| Control | 0 | 100% | 10⁻⁵ | 2220 | 2.2 × 10⁸ |
| Control | 0 | 100% | 10⁻⁶ | 251 | 2.5 × 10⁸ |
| Control | 0 | 100% | 10⁻⁷ | 26 | 2.6 × 10⁸ |
| Chromosome | 0 | 100% | 10⁰ | 0 | 0 |
| Without SDS | 50% | 50% | 10⁰ | 0 | 0 |
| Without SDS | 50% | 50% | 10⁰ | 0 | 0 |
| Without SDS | 50% | 50% | 10⁰ | 0 | 0 |
| With 1% SDS | 50% | 50% | 10⁰ | 0 | 0 |
| With 2% SDS | 50% | 50% | 10⁰ | 0 | 0 |

1 - Control, 0.85% sterile saline
2 - Chromosomal Prep - TE Buffer and Boil
3 - (A, B & C) - 50% Formamide
4 - 50% Formamide, 1.0% SDS
5 - 50% Formamide, 2.0% SDS

### Example 2

### Melting Temperature

A series of melting temperatures were determined according to the conditions shown in Tables 2 and 3. A mismatch in 50% formamide in the DNA strand lowers the melting temperature of the duplex containing LNA-8 from 72 to 66 to 62° C.

As the results in the Tables show, useful melting temperatures are obtained when the oligonucleotides bound in duplex contain at least 15 nucleotide units that have 2′-OMe ribonucleotides as their building blocks. Comparably useful melting temperatures are obtained when the oligonucleotides contain locked nucleic acid (LNA) carbohydrate analogs, as described in [Koshkin et al., 1998]. The data in the Tables also makes clear that the binding between two complementary DNA strands of this length is not useful in 50% formamide, as it is below 30° C. at 50% formamide.

Another metric for utility is a melting temperature of the duplex in an assay below the ambient temperature in a formamide:water mixture whose formamide:water ratio is at equilibrium (remains unchanged over time) upon continues exposure to the ambient humidity. Ambient temperatures are preferably from 0 to 50° C., and ambient humidities are typically between 10 and 90%.

Useful melting temperatures were also obtained when the oligonucleotide was native RNA. Duplexes are also obtained when the standard nucleobases (A, T, G, C) are replaced by a non-standard nucleobase that is independently selected from one of the non-standard nucleobases shown in FIG. 2.

TABLE 2
Melting Temperatures in Formamide With Various
Nucleoside Analogs

| | percent formamide | | | | |
|---|---|---|---|---|---|
| pair \ | 0 | 25 | 50 | 70 | low salt |
| DNA:DNA | 62 | 46 | 29 | | |
| DNA:RNA | 59 | 43 | 30 | | |
| RNA:DNA | 62 | 46 | 34 | | |
| DNA-2′OMe | 61 | 49 | 38 | | |
| 2′-OMe:DNA | 64 | 51 | 40 | | |
| DNA-LNA-5 | 73 | 59 | 47 | 36 | 60 |
| DNA:LNA-8 | | | 70 | | |
| RNA:RNA | 73 | 61 | 49 | | |
| RNA:2′-OMe | 77 | 66 | 59 | | |
| 2′-OMe:RNA | 78 | 68 | 59 | | |
| RNA:LNA-5 | 81 | 70 | 60 | | |

| | | |
|---|---|---|
| strand-1: | 5′-CTT CAG GTA CTG AGT CAA GC-3′ | SEQ ID NO 3 |
| strand-2: | 5′-GCT TGA CTC AGT ACC TGA AG-3′ | SEQ ID NO 4 |
| LNA-5: | 5′-GcT TGA cTc AGT AcC TgA AG-3′ | SEQ ID NO 4 |
| | (cap = DNA; small = LNA) | |

1.5 μM single strand; 100 mM NaCl, 10 mM K-phosphate
pH 7, 0.1 mM EDTA

TABLE 3
Stability of Duplexes in Formamide and Water

| | % | |
|---|---|---|
| Pair | formamide | Tm |
| DNA-DNA | 0 | 61 |
| DNA-RNA | 0 | 58 |
| RNA-DNA | 0 | 62 |
| RNA-RNA | 0 | 73 |
| DNA-DNA | 25 | 44 |
| DNA-RNA | 25 | 42 |
| RNA-DNA | 25 | 45 |
| RNA-RNA | 25 | 60 |
| DNA-DNA | 50 | 28 |
| DNA-RNA | 50 | 29 |
| RNA-DNA | 50 | 31 |
| RNA-RNA | 50 | 48 |
| DNA-DNA | 70 | <20 |
| RNA-RNA | 70 | 40 |
| RNA-RNA | 90 | <25 |

| | |
|---|---|
| 5′-CUUCAGGUACUGAGUCAAGC-3′ | SEQ ID NO 1 |
| 3′-GAAGUCCAUGACUCAGUUCG-5′ | SEQ ID NO 2 |
| 5′-CTT CAG GTA CTG AGT CAA GC-3′ | SEQ ID NO 3 |
| 5′-GCT TGA CTC AGT ACC TGA AG-3′ | SEQ ID NO 4 |
| 5′-GcT TGA cTc AGT AcC TgA AG-3′ | SEQ ID NO 4 |
| (cap = DNA; small = LNA) | |

### REFERENCES

Benner, S. A. (2004) Understanding nucleic acids using synthetic chemistry. Acc. Chem. Res. 37, 784-797.
Blake, R. D., Delcourt, S. G. (1996) Thermodynamic effects of formamide on DNA stability. Nucleic Acids Res. 24, 2095-2103.
Casey, J., Davidson, N. (1977) Rates of formation and thermal stabilities of RNA:DNA and DNA:DNA duplexes at high concentrations of formamide. Nucleic Acids Res. 4, 1539-1552
Chien, Y.-H., Davidson, N. (1978) RNA:DNA hybrids are more stable than DNA:DNA duplexes in concentrated perchlorate ad trichloroacetate solutions. Nucleic Acids Res. 5, 1627-1637.
Koshkin, A. A., Rajwanshi, V. K., Wengel, J. (1998) Novel convenient syntheses of LNA[2.2.1]-bicyclo nucleosides. Tetrahedron Lett. 39, 4381-4384
Duhamel, J., Liu, D. M., Evilia, C., Fleysh, N., Dinter-Gottlieb, G., Lu, P. (1996) Secondary structure content of the HDV ribozyme in 95% formamide. Nucleic Acids Res. 24, 3911-3917.
Hutton, J. R. (1977) Renaturation kinetics and thermal stability of DNA in aqueous solutions of formamide and urea. Nucleic Acids Res. 4, 3537-3555.
Jungmann, R., Liedl, T., Sobey, T. L., Shih, W., Simmel, F. C. (2008) Isothermal assembly of DNA origami structures using denaturing agents. J. Am. Chem. Soc. 130, 10062-10063.
Nam, J.-M., Stoeva, S. I., Mirkin, C. A. (2004) Bio-bar-code-based DNA detection with PCR-like sensitivity, J. Am. Chem. Soc. 126, 5932-5933
Spohr, G., Mirault, M.-E., Imaizumi, T., Scherrer, K. (1976) Molecular weight determination of animal cell RNA by electrophoresis in formamide under fully denaturing conditions on exponential polyacrylamide gels. Eur. J. Biochem. 62, 313-322.
Steger, G. (1994) Thermal denaturation of double stranded nucleic acids. Prediction of temperatures critical for gradient gel electrophoresis and polymerase chain reaction. Nucleic Acids Res. 22, 2760-2768.

What is claimed is:

1. Compositions of matter comprising a mixture containing between 40% to 80% by volume formamide, with the remainder of the liquid volume being 20% to 60% water, wherein said compositions also contain one or more oligonucleotides, wherein at least one oligonucleotides is bound 50% or greater in its duplex form with a complementary oligonucleotide, and wherein the bound oligonucleotide contains at least 15 nucleotides that are 2′-O-alkylribonucleotides or ribonucleotides, or at least five locked nucleotide analogs.

2. The compositions of claim 1 wherein said mixture also contains a detergent.

3. The compositions of claim 2 wherein said detergent is sodium dodecyl sulfate.

4. The compositions of claim 1 wherein said oligonucleotide contains one or more non-standard nucleobases that are may be independently selected from the group consisting of 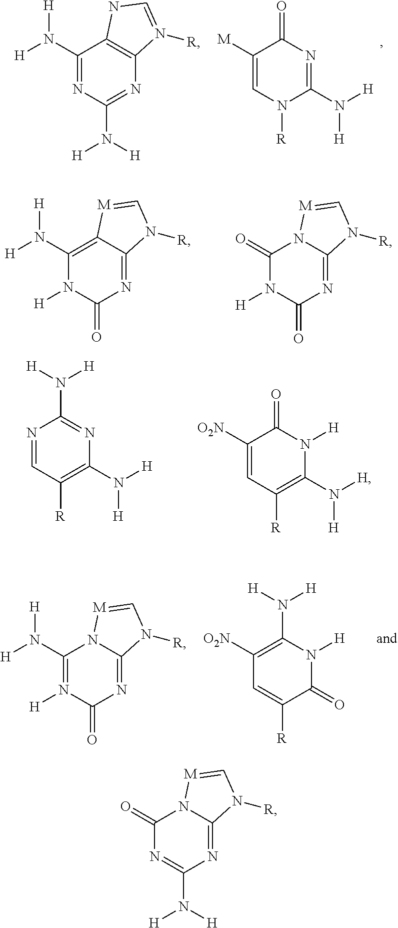
wherein M is selected from the group consisting of N and C-tag, where said tag is selected from the group consisting of fluor, biotin, and alkyl.

5. The compositions of claim 4 wherein said mixture also contains a detergent.

6. The compositions of claim 5 wherein said detergent is sodium dodecyl sulfate.

7. A process for detecting an oligonucleotide that comprises a mixture containing between 40% to 80% by volume formamide, with the remainder of the liquid volume being 20% to 60% water, wherein said compositions also contain one or more oligonucleotides, wherein at least one oligonucleotides is bound 50% or greater in its duplex form with a complementary oligonucleotide, and wherein the bound oligonucleotide contains at least 15 nucleotides that are 2′-O-alkylribonucleotides or ribonucleotides, or at least five locked nucleotide analogs.

8. The process of claim 7 wherein said mixture also contains a detergent.

9. The process of claim 8 wherein said detergent is sodium dodecyl sulfate.

10. The process of claim 7 wherein said oligonucleotide contains one or more non-standard nucleobases that are may be independently selected from the group consisting of 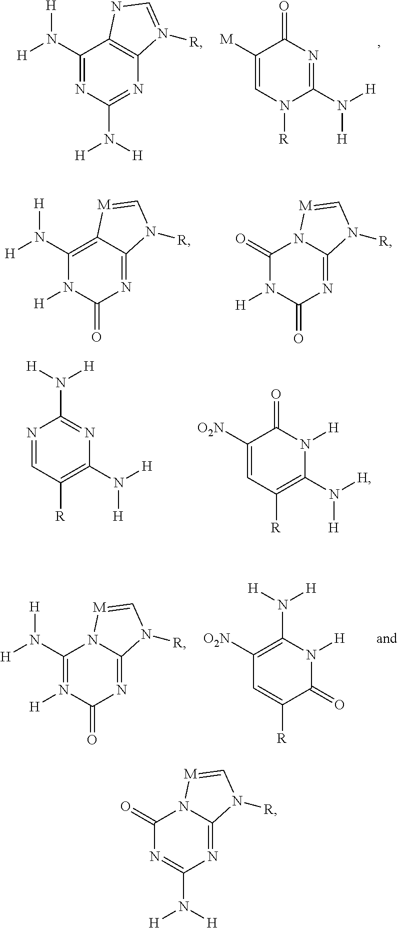
wherein M is selected from the group consisting of N and C-tag, where said tag is selected from the group consisting of fluor, biotin, and alkyl.

11. The compositions of claim 10 wherein said mixture also contains a detergent.

12. The compositions of claim 10 wherein said detergent is sodium dodecyl sulfate.


SEQUENCE LISTING (4 sequences)

SEQ ID NO: 1 - RNA, 20 nt, Artificial Sequence
  Synthetic
cuucagguacugagucaagc

SEQ ID NO: 2 - RNA, 20 nt, Artificial Sequence
  Synthetic
gcuugacucaguaccugaag

SEQ ID NO: 3 - DNA, 20 bp, Artificial Sequence
  Synthetic
cttcaggtactgagtcaagc

SEQ ID NO: 4 - DNA, 20 bp, Artificial Sequence
  Synthetic
gcttgactcagtacctgaag